(19) United States
(12) Patent Application Publication
(10) Pub. No.: US 2002/0031777 A1
(43) Pub. Date: Mar. 14, 2002

(54) Ultra yield amplification reaction

(76) Inventor: Linda Starr-Spires, Milford, NJ (US)

Correspondence Address: THOMAS JEFFERSON UNIVERSITY, 1020 Walnut Street, Philadelphia, PA, 19107, US

(21) Appl. No.: 09/948,336

(22) Filed: Sep. 6, 2001

Related U.S. Application Data
(60) Provisional application No. 60/231,263, filed on Sep. 8, 2000.

(51) Int. Cl. C12Q 1/68; C12P 19/34

(52) U.S. Cl. 435/6; 435/91.2

(57) ABSTRACT

The sensitivity, and therefore specificity, of the polymerase chain reaction is compromised by primer-dimer formation early in the amplification process. Described herein is a simple and novel technique to avoid the formation of primer-dimers. A target nucleic acid is first amplified in a “pre-amplification” reaction, wherein an extremely low concentration of primers bind to the target nucleic acid and not to each other. This allows for the efficient use of the DNA polymerase, deoxynucleoside triphosphates and other reaction components, to extend and amplify the target nucleic acid.

### REFERENCE TO RELATED APPLICATIONS

This application claims priority under 35 U.S.C. §119 based upon U.S. Provisional Patent Application No. 60/231263 filed Sep. 8, 2000.

### GOVERNMENT RIGHTS IN THE INVENTION

This invention was made in part with government support under Grant number AI41399 awarded by the National Institutes of Health. The government has certain rights to the invention.

### FIELD OF THE INVENTION

The present invention relates to the field of molecular biology and, more particularly, to the polymerase chain reaction, wherein a pre-amplification reaction, using concentrations of primers well below those currently being used, eliminates primer-dimer formation and allows the detection of a target nucleic acid.

### BACKGROUND OF THE INVENTION

The polymerase chain reaction (PCR) utilizes the ability of natural or recombinant DNA polymerase enzymes to reproduce a target DNA to high levels. Theoretically, this procedure is capable of producing logarithmic reproductions, (amplification) of a single copy of DNA. However, the sensitivity of the PCR process is compromised by a number of factors during the amplification process, resulting in a significant loss of sensitivity. One of the major problems is the development of a non-specific product during the reaction, commonly known as “primer-dimers”. When these products form, they result in the removal of both primers and deoxyribonucleoside triphosphates (dNTPs) from the reaction, thereby reducing the level of amplification of the desired target and concurrently reducing sensitivity of the reaction.

Primers are designed to be complementary to the desired target, but often exhibit enough homology to each other that they preferentially bind to each other during the polymerase chain reaction (PCR), rather than to the desired target. A standard PCR contains 0.1 to 0.25 μM primers, which is approximately 6-12 trillion copies of each oligonucleotide. While any interaction between these small oligonucleotides would be unstable, as long as greater than one in a trillion of the oligonucleotides is in an “amplifiable” hybridization complex, polymerization will occur, thereby resulting in an appreciable amount of primer-dimer amplification. Because of the short length of these complexes, amplification is very efficient. (Halford, W. P., et al., Analytical Biochemistry, 266181-191, 1999). The use of these relatively high primer concentrations can also result in primers that bind non-specifically to each other and initiate the synthesis of these undesired extension products (primer-dimers) (U.S. Pat. No. 6,001,611).

To overcome the potential for primer-dimer formation during PCR, many researchers add more target or more primer, or both, in order to force the primers to hybridize with the target nucleic acid, rather then each other, thereby forcing the reaction forward. Others amplify the target and use a portion of this amplification to re-amplify the target nucleic acid using either the same master mix or different primers to form a shorter target than the original. The reasoning behind this is to dilute out any non-specific nucleic acids in the original reaction and to further amplify the specific target nucleic acid in the “re-amplified” reaction. Any amplification of non-specific primer extension products will compete with the amplification of the desired target nucleic acid, thereby decreasing the efficiency and yield of the desired nucleic acid.

Those skilled in the art have also tried to eliminate the accumulation of primer-dimers by modifying the conditions of the standard PCR. For instance, some have added a “hotstart” procedure wherein the polymerase, or another critical reagent, is withheld from the reaction until the reaction has reached 90° C. This procedure is thought to increase the sensitivity of the PCR by providing the necessary hybridization specificity. (Chou, Q., et al., Nucleic Acids Res. 20: 1717-1723, 1992; U.S. Pat. No. 6,001,611; Halford, W. P., et al., Analytical Biochemistry, 266181-191, 1999).

Others have added single stranded binding protein to the reaction to non-covalently bind to the primers, thereby preventing hybridization and, thus, primer extension. These single stranded binding proteins are heat labile. When the temperature of the reaction is increased to 90° C.-95° C. for denaturation of nucleic acids, the single stranded binding protein is also denatured. Cooling to 50° C.-60° C. for annealing of the primers to the target nucleic acid is thought to be a sufficiently high temperature to prevent the small oligonucleotide primers from binding to each other while allowing them to bind to the target.

Still others have added DNA polymerase-specific antibodies prior to the start of the reaction. The antibody inhibition of the DNA polymerase is inactivated by a high pre-reaction incubation.

A more cumbersome approach was taken by Brownie, et al. (Brownie, J., et al., Nucleic Acids Res. 25:3235-3241, 1997). These investigators demonstrated a general suppression of primer-dimer formation by adding additional nucleotides, a “tail”, to the 5′ ends of the primers (amplimers). Subsequent amplification of the target nucleic acid uses a different primer that contains a “tag” sequence complementary to the “tail” sequence of the initial primers. The combination of tailed genomic primer and the formation of “pan-handle” structures suppress primer-dimer formation. This method adds extra steps to the PCR, thereby significantly increasing the potential for contamination. Given the sensitivity of the standard PCR, any potential for contamination by even one molecule of nucleic acid jeopardizes the specificity of the reaction. In addition, the use of tailed primers in the initial amplification adds extra, non-target sequences to the product.

To date, those skilled in the art have used modified primers to eliminate, or decrease, any non-specific amplification products. Others have added steps to the PCR in order to increase the sensitivity of the reaction. Those skilled in the art have tried to eliminate primer-dimers by increasing primer concentrations; increasing the target nucleic acid so that the primers can more readily find and bind to the target sequence; increasing the deoxyoligoribonucleic acid concentration (primers); or by increasing the number of denature/anneal/extend cycles.

When competition by non-specific products, most particularly primer-dimers, occurs during PCR the accumulation of specific amplification product stops. Thus, the exponential phase of the amplification reaction reaches a plateau prematurely. The current standard concentrations of primers used are between 0.1 μM and 0.5 μM. (Gelfand, D. H. and Innis, M. A., Optimization of PCRs in PCR Protocols, ed. Innis, M. A., Gelfand, D. H., Sninsky, J. J., and White, T. J., 1990). This excess of oligonucleotide over target nucleic acid creates conditions conducive to the generation of primer-dimers and, thus, the premature termination of amplification of target nucleic acid.

The pre-amplification step of the present invention uses a fraction of this high primer concentration to achieve high levels of amplified target sequence while eliminating primer-dimers. The present invention is a simpler method to reduce and eliminate primer-dimer formation, thereby significantly increasing the sensitivity of the PCR. A “pre-amplification” reaction that begins with an extremely low level of primer, such as 0.0625 μM, along with a limited number of cycles (for example 10 cycles) will allow the primer to be used more efficiently. The kinetics favor the primer binding to the target, which is analogous to increasing the concentration of the target nucleic acid with the exception that use of an initially low primer concentration will favor the primers binding to the target, and not to each other. Primer-dimers are formed typically during the early cycles of PCR, especially when the target nucleic acid concentration is low. In the present invention, the use of extremely low, yet sufficient, concentrations of primers in the early cycles of PCR results in a sensitive and specific nucleic acid amplification reaction.

In the present invention, the reduction and/or elimination of primer-dimers results in the concurrent increase in sensitivity of the PCR since the primers are available to bind to the target, thereby amplifying the target nucleic acid. Detection of low copy numbers of target nucleic acid is critical in diagnostic technology. For instance, the present invention is used for the detection of small numbers of pathogens such as viruses, bacteria, or other microorganisms. A further use of the present invention is in the diagnosis of genetic disorders, as well as the presence of cancerous cells. The addition of the “pre-amplification” step to any PCR-based diagnostic test will increase the sensitivity of the reaction, thereby allowing for the early detection, diagnosis, and treatment of a pathogenic or diseased condition.

### SUMMARY OF THE INVENTION

The present invention is a method for eliminating primer-dimers to enhance the sensitivity in detecting a target nucleic acid. A pre-amplification mix and a sample containing the target nucleic acid are mixed together. A limiting concentration of each of two oligonucleotide primers for the specific sequence being amplified are then added to the pre-amplification mix. Taq DNA polymerase (1-4 units) is added, followed by the denaturing of the target nucleic acid. Denaturation will produce target single stranded nucleic acid molecules. The oligonucleotide primers are then annealed to the target single stranded nucleic acid molecules and extended on these target single stranded nucleic acid molecules. The process from denaturing to extending are repeated a limited number of times.

In one embodiment of the present invention, the concentration of the oligonucleotide primers is no more than 0.0625 μM.

In one embodiment of the invention at least one copy of the target nucleic acid is present.

In one embodiment of the invention the denaturing to extending steps are repeated not more than 10 times.

It is an object of the present invention to add an equal volume of a master mix to the pre-amplification mix, the concentrations of the oligonucleotide primers are increased, and the denaturing to extending steps are repeated for an additional 30-35 times. In one embodiment the concentration of the oligonucleotide primers is increased to a final concentration of between 0.1 and 0.25 μM, preferably 0.25 μM.

It is a further object of the invention that by mixing a sample containing nucleic acid from a patient with a limiting amount of two oligonucleotide primers in the pre-amplification reaction mix the presence of a diseased state is diagnosed.

It is a further object of the invention to present a method for reducing primer-dimers to enhance the sensitivity in detecting a target nucleic acid. A pre-amplification mix and a sample containing the target nucleic acid are mixed together. A limiting concentration of each of two oligonucleotide primers for the specific sequence being amplified are then added to the pre-amplification mix. Taq DNA polymerase (1-4 units) is added, followed by the denaturing of the target nucleic acid. Denaturation will produce target single stranded nucleic acid molecules. The oligonucleotide primers are then annealed to the target single stranded nucleic acid molecules and extended on these target single stranded nucleic acid molecules. The process from denaturing to extending are repeated a limited number of times.

In one embodiment of the present invention, the concentration of the oligonucleotide primers is no more than 0.0625 μM.

In one embodiment of the invention at least one copy of the target nucleic acid is present.

In one embodiment of the invention the denaturing to extending steps are repeated not more than 10 times.

It is an object of the present invention to add an equal volume of a master mix to the pre-amplification mix, the concentrations of the oligonucleotide primers are increased, and the denaturing to extending steps are repeated for an additional 30-35 times. In one embodiment, the concentration of the oligonucleotide primers is increased to a final concentration of between 0.1 and 0.25 μM, preferably 0.25 μM.

### DESCRIPTION OF THE DRAWINGS

FIG. 1. The target input levels, calculated from A₂₆₀ are as follows: lane 1: 100 bp molecular weight marker; lane 2: positive control (undiluted plasmid); lane 3: 7.53×10⁹ plasmid copies per PCR; lane 4: 7.53×10⁷ plasmid copies per PCR; lane 5: 7.53×10⁵ plasmid copies per PCR; lane 6: 7.53×10³ plasmid copies per PCR; lane 7: 7.53×10¹ plasmid copies per PCR; lane 8: 7.53×10⁻¹ plasmid copies per PCR; lane 9: negative control. Ten μL of each amplified sample were loaded in each well for electrophoretic analysis.

FIG. 2. The target input levels, calculated from A₂₆₀ are the following: for the first round amplification: lane 1: 100 bp molecular weight marker; lane 2: positive control; lane 3: 7.53×10⁹ plasmid copies per PCR; lane 4: 7.53×10⁷ plasmid copies per PCR; lane 5: 7.53×10⁵ plasmid copies per PCR; lane 6: 7.53×10³ plasmid copies per PCR; lane 7: 7.53×10¹ plasmid copies per PCR; lane 8: 7.53×10⁻¹ plasmid copies per PCR; lane 9: negative control. For the second round of amplification the target input levels are the following: lane 10: positive control; lane 11: 7.53×10⁹ plasmid copies per PCR; lane 12: 7.53×10⁷ plasmid copies per PCR; lane 13: 7.53×10⁵ plasmid copies per PCR; lane 14: 7.53×10³ plasmid copies per PCR; lane 15: 7.53×10¹ plasmid copies per PCR; lane 16: 7.53×10⁻¹ plasmid copies per PCR; lane 17: negative control; and lane 18: 100 bp molecular weight marker.

### DESCRIPTION OF THE INVENTION

There is a long felt need to increase the sensitivity of PCR to allow the detection of single copy nucleic acid. The current methodology either increases one or more components of the reaction or adds a number of steps to the PCR to increase sensitivity. The present invention allows a “pre-amplification” step using limiting concentrations of primers to amplify one or more copies of nucleic acid. Following this limited number of denature/anneal/amplify cycles a master mix containing all of the PCR components with the standard amount of primers, generally a final concentration of 0.25 μM, is added, and a second amplification is carried out. Any contamination of the samples during the addition of the second master mix is eliminated by automated handling equipment that adds the second master mix via cap-piercing devices. Further, adaptation to currently available high-throughput equipment allows for many samples to be analyzed using a multi-well format.

Each polymerase chain reaction will contain the following pre-amplification mix: 10-50 mM Tris-HCl, between pH 8.3 and 8.8; 0.5-2.5 mM MgCl₂; 0-50 mM KCl; 20-200 μM dNTP. The target nucleic acid samples, 0.02-0.0625 μM primers and 1-4 units of Taq DNA polymerase are added to this master mix. Optionally, gelatin (up to 0.001%), bovine serum albumin (up to 100 μg/ml) or nonionic detergents such as Tween 20 or Laureth 12 (0.05-0.1%) can be included to help stabilize the polymerase. All of the ingredients are mixed and PCR is carried out for up to 10 cycles. While PCR cycle conditions will vary depending on each specific target nucleic acid/primer combination, in general, each cycle consists of a denaturation at 90° C.-95° C. for 15-30 seconds, primer annealing at a temperature 3° C.-5° C. below the true Tₘ of the amplification primers and extension of primers on the target nucleic acid template for 30 seconds to one minute (one minute for every 1000 bp) at 72° C. This procedure is carried out in a DNA Thermal Cycler.

The standard methods for optimizing the annealing temperature, polymerase concentration, and buffer constituents for PCR for a particular target sequence and a particular set of primers are well known to those skilled in the art. (U.S. Pat. Nos. 4,683,195; 4,683,202; Saiki et al., Science 230:1350-1354, 1985; Mullis et al., Cold Springs Harbor Symp. Quant. Biol., 51:263-273, 1986; and Mullis and Faloona, Methods Enzymol., 155:335-350, 1987; each of which is incorporated herein by reference).

Following the pre-amplification reaction, 50 μl of a master mix is added to each reaction tube. The master mix contains: 10-50 mM Tris-HCl, between pH 8.3 and 8.8; 0.5-2.5 mM MgCl₂; 0-50 mM KCl; 20-200 μM dNTP. Again, it is optional to include gelatin (up to 0.001%), bovine serum albumin (up to 100 μg/ml) or nonionic detergents such as Tween 20 or Laureth 12 (0.05-0.1%) to help stabilize the polymerase. The same oligonucleotide primers are added to this second reaction mix to achieve a final concentration of 0.1-0.25 μM, most preferably 0.25 μM. Additional Taq DNA polymerase is optional, as the polymerase added to the initial pre-amplification reaction is sufficient to continue the additional 30-35 cycles of amplification.

At the completion of the 30-35 denaturation/annealing/amplification cycles the reaction is analyzed for the presence of target nucleic acid. Detection of the amplified product is by agarose gel electrophoresis, polyacrylamide gel electrophoresis, chromatography, Southern Blot analysis, Dot Blot analysis, or any other means that are well known to those skilled in the art. Those skilled in the art can select a suitable analysis method depending on that particular situation.

The method of the present invention enables detection and characterization of specific nucleic acid sequences. In one embodiment of the invention, sequences associated with any infectious disease, genetic disorder, or cellular disorder, such as cancer, are detected. The enhanced sensitivity of the pre-amplification method of PCR is also useful for detection of nucleic acids in small samples, for example, in forensic medicine. Samples that are used for detecting a nucleic acid include, but are not limited to, blood or a blood component, any body fluid (such as urine, semen, cerebrospinal fluid etc.), tissue, hair, any cell, clothing, or any item that is suspected of containing a nucleic acid.

Pre-amplification of a target nucleic acid is a reliable approach for eliminating pirmer-dimers, with the subsequent enhanced sensitivity for detecting one or more copies of a target nucleic acid. The ability to detect such small amounts of nucleic acid will aid in the diagnosis of, and therapeutic approach to, any disorder associated with the presence of or alteration of a nucleic acid.

Primer-dimer Formation Under Standard PCR Conditions

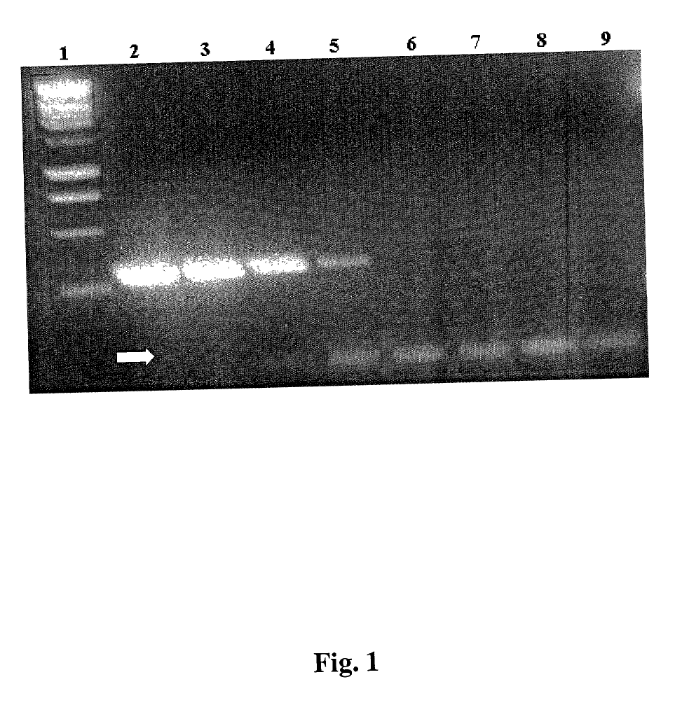

The PCR reaction in FIG. 1 was run using standard conditions: 0.250 μM each primer and 250 μM deoxyribonucleotides (dNTPs) in a 50 μL reaction. The target DNA was a plasmid containing a single copy of the SV40 viral genome. The amplification profile had been optimized for this primer pair and was designed to amplify an approximately 300 base pair region of the T Antigen gene of the SV40. The amplification profile consisted of an initial denaturation of plasmid DNA at 95° C. for 10 minutes to insure complete denaturation of the plasmid. The amplification profile of 45 cycles is as follows: denature at 94° C. for 1 minute; anneal at 53° C. for 1 minute; extension at 72° C. for 1 minute. A single final extension of 72° C. for 3 minutes was used to allow complete extension of any unfinished product from previous amplification rounds.

In amplifications where there is insufficient target DNA to which the primers can hybridize (FIG. 1, lanes 2-9), the primers will hybridize to themselves or each other. The Taq polymerase indiscriminately extends these non-specific hybridization products resulting in the formation of “primer-dimers” (FIG. 1, arrow). In subsequent rounds, primer-dimers then serve as templates, continuing to utilize primers and dNTPs, thereby permanently removing these components from the amplification process. This non-specific amplification significantly impairs the ability of the PCR process to produce the desired product, thus reducing the overall sensitivity of the method. Under these conditions, the lowest detectable plasmid DNA level is approximately 7.53×10⁵ copies per PCR (FIG. 1, lane 5).

Effect of Differential Primer Concentrations on PCR Amplification Yield

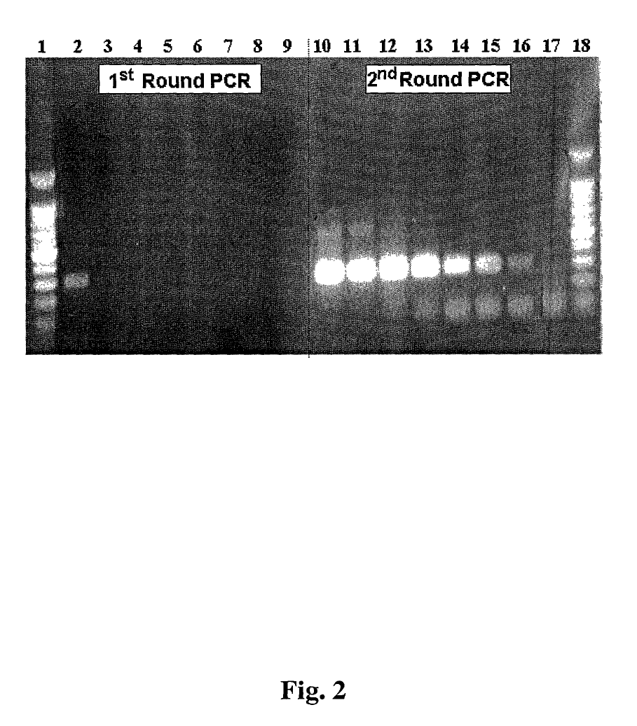

In the first round of amplification (FIG. 2, lanes 2-9), the primer concentration is reduced to 0.0625 μM. The primers and target DNA were the same as that used in FIG. 1. All other components are at the same concentrations as standard PCR (supra). The denaturation of plasmid DNA was at 95° C. for 10 minutes followed by 10 cycles of amplification. The amplification profile in the first round is as follows: denaturation at 94° C. for 1 minute; anneal at 53° C. for 1 minute; extend at 72° C. for 1 minute; and a final extension at 72° C. for 3 minutes. Before beginning the second round of amplification, 10 μL of each sample was retained for analysis. To begin the second round of amplification (FIG. 2, lanes 12-17), 50 μL of master mix (supra) was added to each sample. The master mix for this round included all components required for amplification, with primer concentrations increased to 0.250 μM in the final reaction. The amplification profile for the second round was as follows: denature at 94° C. for 1 minute; anneal at 53° C. for 1 minute; extend at 72° C. for 1 minute for 35 cycles. A single final extension of 72° C. for 3 minutes was used to allow complete extension of any unfinished product from the previous amplification rounds. Ten μL of each product from both rounds of amplification were loaded per well for electrophoretic analysis (FIG. 2).

The combination of the lower primer concentration used in the first round of PCR (FIG. 2, lanes 2-8) and re-amplification with higher primer concentrations in the second round of PCR results in significantly reduced primer-dimer formation and a concomitant increase in the desired product (FIG. 2, lanes 10-16). Under these conditions, the lowest detectable level of plasmid DNA is approximately one copy per PCR (FIG. 2, lane 16). Negative samples from the first and second rounds (FIG. 2, lanes 9 and 17, respectively) exhibit no detectable contaminant, thereby indicating that the addition of the second round master mix did not result in the addition of exogenous template.

What is claimed is:

1. A method for eliminating primer-dimers for enhanced sensitivity in detecting a target nucleic acid, comprising:
a) mixing together a pre-amplification mix and a sample with said target nucleic acid;
b) adding to said pre-amplification mix a limiting concentration of each of two oligonucleotide primers for a specific sequence being amplified;
c) adding to said pre-amplification mix of step b) 1-4 units of a Taq DNA polymerase;
d) denaturing said target nucleic acid to produce target single stranded nucleic acid molecules;
e) annealing said oligonucleotide primers to said target single stranded nucleic acid molecules;
f) extending said oligonucleotide primers on said target single stranded nucleic acid molecules from step e); and
g) repeating steps d) to f) for a limited number of times.

2. The method of claim 1, comprising a concentration of said oligonucleotide primers of no more than 0.0625 μM.

3. The method of claim 1, comprising at least one copy of said target nucleic acid.

4. The method of claim 1, comprising repeating steps d) to f) not more than 10 times.

5. The method of claim 1, further comprising:
a) adding an equal volume of a master mix to said pre-amplification mix;
b) increasing concentrations of said oligonucleotide primers; and
c) repeating steps d) to f) for an additional 30-35 times.

6. The method of claim 5, comprising increasing said concentration of said oligonucleotide primers to a final concentration of between 0.1 and 0.25 μM.

7. A method of diagnosing the presence of a diseased state, comprising mixing a sample containing nucleic acid from a patient with a limiting amount of two oligonucleotide primers in said pre-amplification reaction mix of claim 1.

8. A method for reducing primer-dimers for enhanced sensitivity in detecting a target nucleic acid, comprising:
a) mixing together a pre-amplification mix and a sample with said target nucleic acid;
b) adding to said pre-amplification mix a limiting concentration of each of two oligonucleotide primers for a specific sequence being amplified;
c) adding to said pre-amplification mix of step b) 1-4 units of a Taq DNA polymerase;
d) denaturing said target nucleic acid to produce target single stranded nucleic acid molecules;
e) annealing said oligonucleotide primers to said target single stranded nucleic acid molecules;
f) extending said oligonucleotide primers on said target single stranded nucleic acid molecules from step e); and
g) repeating steps d) to f) for a limited number of times.

9. The method of claim 8, comprising a concentration of said oligonucleotide primers of no more than 0.0625 μM.

10. The method of claim 8, comprising at least one copy of said target nucleic acid.

11. The method of claim 8, comprising repeating steps d) to f) not more than 10 times.

12. The method of claim 8, further comprising:
h) adding an equal volume of a master mix to said pre-amplification mix;
i) increasing concentrations of said oligonucleotide primers; and
j) repeating steps d) to f) for an additional 30-35 times.

13. The method of claim 12, comprising increasing said concentration of said oligonucleotide primers to a final concentration of between 0.1 and 0.25 μM.
